(19) United States
(12) Patent Application Publication
(10) Pub. No.: US 2021/0380958 A1
(43) Pub. Date: Dec. 9, 2021

(54) PROCESS FOR ENZYMATIC HYDROLYSIS OF CARBOHYDRATE MATERIAL AND FERMENTATION OF SUGARS

(71) Applicant: DSM IP ASSETS B.V, Heerlen (NL)

(72) Inventors: Maaike APPELDOORN, Echt (NL); Bertus NOORDAM, Echt (NL)

(21) Appl. No.: 17/287,227

(22) PCT Filed: Oct. 23, 2019
(86) PCT No.: PCT/EP2019/078783

§ 371 Date: Apr. 21, 2021

(30) Foreign Application Priority Data
Oct. 24, 2018 (EP) 18202215.2
Apr. 2, 2019 (EP) 19166813.6

(51) Int. Cl. C12N 9/42; C12P 19/02; C12P 19/14; C13K 1/02

(52) U.S. Cl. CPC C12N 9/2437; C12Y 302/01004; C12P 19/02; C12P 2203/00; C13K 1/02; C12P 2201/00; C12P 19/14

(57) ABSTRACT

The invention relates to a process for the preparation of an enzyme composition from cellulosic material.

### FIELD

The application relates to a process for the preparation of an enzyme composition.

### BACKGROUND

Lignocellulosic material is primarily composed of cellulose, hemicellulose and lignin and provides an attractive platform for generating alternative energy sources to fossil fuels. The material is available in large amounts and can be converted into valuable products e.g. sugars or biofuel, such as bioethanol.

Producing fermentation products from lignocellulosic material is known in the art and generally includes the steps of pretreatment, hydrolysis, fermentation, and optionally recovery of the fermentation products.

Commonly, the sugars produced are converted into valuable fermentation products such as ethanol by microorganisms like yeast. The fermentation takes place in a separate, preferably anaerobic, process step, either in the same or in a different vessel.

In general, cost of enzyme production is a major cost factor in the overall production process of fermentation products from lignocellulosic material. Thus far, reduction of enzyme production costs is achieved by applying enzyme products from a single or from multiple microbial sources (see WO 2008/008793) with broader and/or higher (specific) hydrolytic activity. This leads to a lower enzyme need, faster conversion rates and/or higher conversion yields and thus to lower overall production costs.

Next to the optimization of enzymes, optimization of process design is a crucial tool to reduce overall costs of the production of sugar products and fermentation products. For example, sugar loss by means of sugar degradation products increases with decreasing yield. Since sugar degradation products can inhibit fermentation, process design should be optimized to decrease the amount of these sugar degradation products.

For economic reasons, it is therefore desirable to include new and innovative process configurations aimed at reducing overall production costs in the process involving pretreatment, hydrolysis and fermentation of carbohydrate material.

### SUMMARY

An object of the application is to provide an improved process for the preparation of an enzyme composition. The process is improved by using specific hydrolysis conditions.

### DETAILED DESCRIPTION

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows. The articles “a” and “an” are used herein to refer to one or to more than one (i.e. to one or at least one) of the grammatical object of the article. By way of example, “an element” may mean one element or more than one element.

Described herein is a process for the preparation of an enzyme composition, comprising the steps of (a) pretreating cellulosic material, (b) enzymatically hydrolysing the pretreated cellulosic material to obtain a hydrolysate, (c) fermenting the hydrolysate to produce the enzyme composition, and (d) optionally, recovering the enzyme composition, wherein the pH of the pretreated cellulosic material is controlled before and/or during step (b) by adding a hydroxide of an alkali metal and/or a hydroxide of an alkaline earth metal to the pretreated cellulosic material.

Described herein is a process for the preparation of an enzyme composition from cellulosic material, comprising the steps of (a) pretreating cellulosic material, (b) enzymatically hydrolysing the pretreated cellulosic material to obtain a hydrolysate, (c) fermenting the hydrolysate to produce the enzyme composition, and (d) optionally, recovering the enzyme composition, wherein the pH of the pretreated cellulosic material is controlled before and/or during step (b) by adding a hydroxide of an alkali metal and/or a hydroxide of an alkaline earth metal to the pretreated cellulosic material.

In a preferred embodiment the pH of the pretreated cellulosic material is controlled during step (b) by adding a hydroxide of an alkali metal and/or a hydroxide of an alkaline earth metal to the pretreated cellulosic material. Instead of the term “hydrolysate”, the term “sugar product”, “one or more sugars” or “sugar” can be used.

Described herein is also a process for the preparation of an enzyme composition, comprising the steps of (a) pretreating cellulosic material, (b) enzymatically hydrolysing the pretreated cellulosic material to obtain a hydrolysate, (c) fermenting the hydrolysate to produce the enzyme composition, and (d) optionally, the enzyme composition, wherein the pH of the pretreated cellulosic material is controlled before and/or during step (b) by adding a strong base to the pretreated cellulosic material. In a preferred embodiment the pH of the pretreated cellulosic material is controlled during step (b) by adding a strong base to the pretreated cellulosic material. The enzymatic hydrolysis step (b) can be done with any of the enzyme compostions as described herein

As used herein, “the pH of the pretreated cellulosic material is controlled before step (b)” means that the pH is controlled after the pretreatment step has ended and before the hydrolysis step has started. In other words, the hydroxide of an alkali metal and/or a hydroxide of an alkaline earth metal or the strong base is added after the pretreatment step has ended and before the hydrolysis step has started.

In a preferred embodiment the obtained hydrolysate is concentrated before fermentation. Concentration can be done by standard methods such as evaporation, centrifugation, filtration, sedimentation or any combination thereof.

In a preferred embodiment the obtained hydrolysate is sterilized before fermentation. Sterilization can be done by standard methods such as heat treatment, sterile filtration or any combination thereof.

The obtained hydrolysate can be first sterilized and then concentrated, but preferably the obtained hydrolysate is concentrated and then the concentrated hydrolysate is sterilized.

In an embodiment the obtained hydrolysate can be subjected to a preservation step. This step can be performed before, during or after the concentration step and/or before, during or after the sterilization step.

In an embodiment the pretreatment step and/or the hydrolysis step are done in a reactor. In an embodiment the pretreatment step and/or the hydrolysis step may also be done in two, three, four, five, six, seven, eight, nine, ten or even more reactors. So, the term “reactor” is not limited to a single reactor but may mean multiple reactors. In an embodiment the pretreatment step and the hydrolysis step are performed in different reactors.

In the processes as described herein, pretreated cellulosic material may be added to the reactor in which the hydrolysis step takes place. This can be done batch-wise, fed-batch wise or continuously. In an embodiment an enzyme composition is added to the reactor in which the hydrolysis step takes place. This can be done batch-wise, fed-batch wise or continuously. The enzyme composition may be an aqueous composition.

In an embodiment the hydrolysis step comprises a liquefaction step and a saccharification step. In an embodiment the liquefaction step and the saccharification step each can be done in a single reactor, but each may also be done in multiple reactors. In an embodiment the liquefaction step and the saccharification step are done in different reactors.

In an embodiment the pretreatment is done in a reactor having a volume of 10-500 m³, preferably 30-200 m³, more preferably of 100-150 m³. In case multiple reactors are used in the pretreatment of the processes as described herein, they may have the same volume, but also may have a different volume.

In an embodiment the pretreatment reactor used in the processes as described herein has a ratio height to diameter of 3:1 to 12:1.

In an embodiment the hydrolysis step is done in a reactor having a volume of at least 10 m³. In an embodiment the hydrolysis step is done in a reactor having a volume of 10-5000 m³, preferably of 50-5000 m³. In case multiple reactors are used in the hydrolysis step, they may have the same volume, but also may have a different volume.

In an embodiment the reactor in which the hydrolysis step is done has a ratio height to diameter of to 0.1:1 to 10:1.

In an embodiment oxygen is added to the pretreated cellulosic material during the hydrolysis step. In an embodiment oxygen is added during at least a part of the hydrolysis step. Oxygen can be added continuously or discontinuously during the hydrolysis step. In an embodiment oxygen is added one or more times during the processes as described herein. In an embodiment oxygen is added to the reactors used in the hydrolysis step.

Oxygen can be added in several forms. For example, oxygen can be added as oxygen gas, oxygen-enriched gas, such as oxygen-enriched air, or air. Oxygen may also be added by means of in situ oxygen generation.

Examples how to add oxygen include, but are not limited to, addition of oxygen by means of sparging, blowing, electrolysis, chemical addition of oxygen, filling a reactor used in the hydrolysis step from the top (plunging the liquefied hydrolysate into the reactor and consequently introducing oxygen into the hydrolysate) and addition of oxygen to the headspace of a reactor. When oxygen is added to the headspace of the reactor, sufficient oxygen necessary for the hydrolysis reaction may be supplied. In general, the amount of oxygen added to the reactor can be controlled and/or varied. Restriction of the oxygen supplied is possible by adding only oxygen during part of the hydrolysis time in the reactor. Another option is adding oxygen at a low concentration, for example by using a mixture of air and recycled air (air leaving the reactor) or by “diluting” air with an inert gas. Increasing the amount of oxygen added can be achieved by addition of oxygen during longer periods of the hydrolysis time, by adding the oxygen at a higher concentration or by adding more air. Another way to control the oxygen concentration is to add an oxygen consumer and/or an oxygen generator. Oxygen can be introduced into the pretreated carbohydrate material present in the reactor. It can also be introduced into the headspace of the reactor. Oxygen can be blown into the pretreated cellulosic material present in the reactor. It can also be blown into the headspace of the reactor.

In an embodiment oxygen is added to the reactor used in the hydrolysis step before and/or during and/or after the addition of the pretreated cellulosic material to the reactor. The oxygen may be introduced together with the pretreated cellulosic material that enters the reactor. The oxygen may be introduced into the material stream that will enter the reactor or with part of the reactor contents that passes an external loop of the reactor. Preferably, oxygen is added when the pretreated cellulosic material is present in the reactor.

In an embodiment oxygen is added during the hydrolysis step to keep the dissolved oxygen at 11% to 80% of the saturation level. In an embodiment oxygen is added during the hydrolysis step to keep the dissolved oxygen at 20% to 60% of the saturation level.

In an embodiment the hydroxide of an alkali metal and/or the hydroxide of an alkaline earth metal are selected from the group consisting of aluminium hydroxide, barium hydroxide, calcium hydroxide, caesium hydroxide, potassium hydroxide, lithium hydroxide, magnesium hydroxide, sodium hydroxide, rubidium hydroxide, strontium hydroxide and any combination thereof. In a preferred embodiment the hydroxide of an alkali metal and/or the hydroxide of an alkaline earth metal are selected from the group consisting of calcium hydroxide, sodium hydroxide and potassium hydroxide.

In an embodiment the strong base is selected from the group consisting of barium hydroxide, calcium hydroxide, caesium hydroxide, potassium hydroxide, lithium hydroxide, magnesium hydroxide, sodium hydroxide, rubidium hydroxide, strontium hydroxide and any combination thereof. In a preferred embodiment the strong base is selected from the group consisting of calcium hydroxide, sodium hydroxide and potassium hydroxide.

In an embodiment the pH of the pretreated cellulosic material is controlled before and/or during step (b) (i.e. the hydrolysis step) such that is from 3.0 to 6.5. Preferably, it is from 3.5 to 5.5, more preferably it is from 4.0 to 5.0. Preferably, the pH is controlled during step (b).

In an embodiment the pH is measured before and/or during step (b). Preferably, the pH is controlled during step (b) and when the pH is outside the preferred range the hydroxide of an alkali metal and/or the hydroxide of an alkaline earth metal or the strong base is added to the pretreated cellulosic material.

In an embodiment the enzyme composition is from a fungus, preferably a filamentous fungus. In an embodiment the enzyme composition is produced by a fungus, preferably a filamentous fungus. In an embodiment the enzymes in the enzyme composition are derived from a fungus, preferably a filamentous fungus. In an embodiment the enzyme composition comprises a fungal enzyme, preferably a filamentous fungal enzyme. In an embodiment step (c) of the processes as herein described comprises fermenting the hydrolysate by a fungus to produce the enzyme composition.

“Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). Filamentous fungi include, but are not limited to Acremonium, Agaricus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes pleurotus, Trichoderma and Trichophyton. In a preferred embodiment the fungus is Rasamsonia, with Rasamsonia emersonii being most preferred. Ergo, the processes as described herein are advantageously applied in combination with enzymes derived from a microorganism of the genus Rasamsonia or the enzymes used in the processes as described herein comprise a Rasamsonia enzyme.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

Preferably, the processes as described herein are done with thermostable enzymes. “Thermostable” enzyme as used herein means that the enzyme has a temperature optimum of 50° C. or higher, 60° C. or higher, 70° C. or higher, 75° C. or higher, 80° C. or higher, or even 85° C. or higher. They may for example be isolated from thermophilic microorganisms or may be designed by the skilled person and artificially synthesized. In one embodiment the polynucleotides encoding the thermostable enzymes may be isolated or obtained from thermophilic or thermotolerant filamentous fungi or isolated from non-thermophilic or non-thermotolerant fungi but are found to be thermostable. By “thermophilic fungus” is meant a fungus that grows at a temperature of 50° C. or higher. By “themotolerant” fungus is meant a fungus that grows at a temperature of 45° C. or higher, having a maximum near 50° C.

Suitable thermophilic or thermotolerant fungal cells may be Humicola, Rhizomucor, Myceliophthora, Rasamsonia, Talaromyces, Thermomyces, Thermoascus or Thielavia cells, preferably Rasamsonia cells. Preferred thermophilic or thermotolerant fungi are Humicola grisea var. thermoidea, Humicola lanuginosa, Myceliophthora thermophila, Papulaspora thermophilia, Rasamsonia byssochlamydoides, Rasamsonia emersonii, Rasamsonia argillacea, Rasamsonia eburnean, Rasamsonia brevistipitata, Rasamsonia cylindrospora, Rhizomucor pusillus, Rhizomucor miehei, Talaromyces bacillisporus, Talaromyces leycettanus, Talaromyces thermophilus, Thermomyces lenuginosus, Thermoascus crustaceus, Thermoascus thermophilus Thermoascus aurantiacus and Thielavia terrestris.

Rasamsonia is a new genus comprising thermotolerant and thermophilic Talaromyces and Geosmithia species. Based on phenotypic, physiological and molecular data, the species Talaromyces emersonii, Talaromyces byssochlamydoides, Talaromyces eburneus, Geosmithia argillacea and Geosmithia cylindrospora were transferred to Rasamsonia gen. nov. Talaromyces emersonii, Penicillium geosmithia emersonii and Rasamsonia emersonii are used interchangeably herein.

In the processes as described herein enzyme compositions are used. In an embodiment the compositions are stable. “Stable enzyme compositions” as used herein means that the enzyme compositions retain activity after 30 hours of hydrolysis reaction time, preferably at least 10%, 20%, 30%, 40%, 50%, 60%, 65%, 70%, 75%, 80% 85%, 90%, 95%, 96%, 97%, 98%, 99% or 100% of its initial activity after 30 hours of hydrolysis reaction time. In an embodiment the enzyme composition retains activity after 40, 50, 60, 70, 80, 90 100, 150, 200, 250, 300, 350, 400, 450, 500 hours of hydrolysis reaction time.

The enzymes may be prepared by fermentation of a suitable substrate with a suitable microorganism, e.g. Rasamsonia emersonii or Aspergillus niger, wherein the enzymes are produced by the microorganism. The microorganism may be altered to improve or to make the enzymes. For example, the microorganism may be mutated by classical strain improvement procedures or by recombinant DNA techniques. Therefore, the microorganisms mentioned herein can be used as such to produce the enzymes or may be altered to increase the production or to produce altered enzymes which might include heterologous enzymes, e.g. cellulases, thus enzymes that are not originally produced by that microorganism. Preferably, a fungus, more preferably a filamentous fungus is used to produce the enzymes. Advantageously, a thermophilic or thermotolerant microorganism is used. Optionally, a substrate is used that induces the expression of the enzymes by the enzyme producing microorganism.

The enzymes are used to hydrolyse the pretreated cellulosic material (release sugars from cellulosic material that comprises polysaccharides). Cellulosic materials as used herein comprise polysaccharides. The polysaccharides can be celluloses (glucans) and hemicelluloses (xylans, heteroxylans and xyloglucans). In addition, some hemicellulose may be present as glucomannans, for example in wood-derived carbohydrate material. The enzymatic hydrolysis of these polysaccharides to soluble sugars, including both monomers and multimers, for example glucose, cellobiose, xylose, arabinose, galactose, fructose, mannose, rhamnose, ribose, galacturonic acid, glucuronic acid and other hexoses and pentoses occurs under the action of different enzymes acting in concert. A sugar product comprises soluble sugars, including both monomers and multimers. In an embodiment the sugar product comprises glucose, galactose and arabinose. Examples of other sugars are cellobiose, xylose, arabinose, galactose, fructose, mannose, rhamnose, ribose, galacturonic acid, glucoronic acid and other hexoses and pentoses. The sugar product may be used as such or may be further processed for example recovered and/or purified.

In addition, cellulosic materials may comprise pectins and other pectic substances such as arabinans, which may make up considerably proportion of the dry mass of typically cell walls from non-woody plant tissues (about a quarter to half of dry mass may be pectins). Furthermore, the cellulosic material may comprise lignin.

Enzymes that may be used in the processes as described herein are described in more detail below.

Lytic polysaccharide monooxygenases, endoglucanases (EG) and exo-cellobiohydrolases (CBH) catalyze the hydrolysis of insoluble cellulose to products such as cellooligosaccharides (cellobiose as a main product), while β-glucosidases (BG) convert the oligosaccharides, mainly cellobiose and cellotriose, to glucose.

Xylanases together with other accessory enzymes, for example α-L-arabinofuranosidases, feruloyl and acetylxylan esterases, glucuronidases, and β-xylosidases catalyze the hydrolysis of hemicellulose.

An enzyme composition for use in the processes as described herein may comprise at least two activities, although typically a composition will comprise more than two activities, for example, three, four, five, six, seven, eight, nine or even more activities. Typically, an enzyme composition for use in the processes as described herein comprises at least two cellulases. The at least two cellulases may contain the same or different activities. The enzyme composition for use in the processes as described herein may also comprises at least one enzyme other than a cellulase. Preferably, the at least one other enzyme has an auxiliary enzyme activity, i.e. an additional activity which, either directly or indirectly leads to lignocellulose degradation. Examples of such auxiliary activities are mentioned herein and include, but are not limited, to hemicellulases.

An enzyme composition for use in the processes as described herein at least comprises a lytic polysaccharide monooxygenase (LPMO), an endoglucanase (EG), a cellobiohydrolase (CBH), an endoxylanase (EX), a beta-xylosidase (BX) and a beta-glucosidase (BG). An enzyme composition may comprise more than one enzyme activity per activity class. For example, a composition may comprise two endoglucanases, for example an endoglucanase having endo-1,3(1,4)-β glucanase activity and an endoglucanase having endo-β-1,4-glucanase activity.

A composition for use in the processes as described herein may be derived from a fungus, such as a filamentous fungus, such as Rasamsonia, such as Rasamsonia emersonii. In an embodiment at least one of enzymes may be derived from Rasamsonia emersonii. If needed, the enzyme can be supplemented with additional enzymes from other sources. Such additional enzymes may be derived from classical sources and/or produced by genetically modified organisms.

In addition, enzymes in the enzyme compositions for use in the processes as described herein may be able to work at low pH. For the purposes of this invention, low pH indicates a pH of 5.5 or lower, 5 or lower, 4.9 or lower, 4.8 or lower, 4.7 or lower, 4.6 or lower, 4.5 or lower, 4.4 or lower, 4.3 or lower, 4.2 or lower, 4.1 or lower, 4.0 or lower 3.9 or lower, 3.8 or lower, 3.7 or lower, 3.6 or lower, 3.5 or lower.

The enzyme composition for use in the processes as described herein may comprise a cellulase and/or a hemicellulase and/or a pectinase from Rasamsonia. They may also comprise a cellulase and/or a hemicellulase and/or a pectinase from a source other than Rasamsonia. They may be used together with one or more Rasamsonia enzymes or they may be used without additional Rasamsonia enzymes being present.

An enzyme composition for use in the processes as described herein may comprise a lytic polysaccharide monooxygenase (LPMO), an endoglucanase (EG), a cellobiohydrolase I (CBHI), a cellobiohydrolase II (CBHII), a beta-glucosidase (BG), an endoxylanase (EX) and a beta-xylosidase (BX).

An enzyme composition for use in the processes as described herein may comprise one type of cellulase activity and/or hemicellulase activity and/or pectinase activity provided by a composition as described herein and a second type of cellulase activity and/or hemicellulase activity and/or pectinase activity provided by an additional cellulase/hemicellulase/pectinase.

In an embodiment the enzyme composition comprises a whole fermentation broth of a fungus. In an embodiment said broth comprises an endoglucanase, a cellobiohydrolase, a beta-glucosidase, a endoxylanase, a beta-xylosidase and a lytic monosaccharide oxygenase. These enzymes have been described in more detail herein.

As used herein, a cellulase is any polypeptide which is capable of degrading or modifying cellulose. A polypeptide which is capable of degrading cellulose is one which is capable of catalyzing the process of breaking down cellulose into smaller units, either partially, for example into cellodextrins, or completely into glucose monomers. A cellulase as described herein may give rise to a mixed population of cellodextrins and glucose monomers. Such degradation will typically take place by way of a hydrolysis reaction.

As used herein, a hemicellulase is any polypeptide which is capable of degrading or modifying hemicellulose. That is to say, a hemicellulase may be capable of degrading or modifying one or more of xylan, glucuronoxylan, arabinoxylan, glucomannan and xyloglucan. A polypeptide which is capable of degrading a hemicellulose is one which is capable of catalyzing the process of breaking down the hemicellulose into smaller polysaccharides, either partially, for example into oligosaccharides, or completely into sugar monomers, for example hexose or pentose sugar monomers. A hemicellulase as described herein may give rise to a mixed population of oligosaccharides and sugar monomers. Such degradation will typically take place by way of a hydrolysis reaction.

As used herein, a pectinase is any polypeptide which is capable of degrading or modifying pectin. A polypeptide which is capable of degrading pectin is one which is capable of catalyzing the process of breaking down pectin into smaller units, either partially, for example into oligosaccharides, or completely into sugar monomers. A pectinase as described herein may give rise to a mixed population of oligosacchardies and sugar monomers. Such degradation will typically take place by way of a hydrolysis reaction.

Accordingly, an enzyme composition for use in the processes as described herein may comprise one or more of the following enzymes, a lytic polysaccharide monooxygenase (e.g. GH61), a cellobiohydrolase, an endo-β-1,4-glucanase, a beta-glucosidase, and a β-(1,3)(1,4)-glucanase. A composition for use in the processes as described herein may also comprise one or more hemicellulases, for example, an endoxylanase, a β-xylosidase, a α-L-arabionofuranosidase, an α-D-glucuronidase, an acetyl xylan esterase, a feruloyl esterase, a coumaroyl esterase, an α-galactosidase, a β-galactosidase, a β-mannanase and/or a β-mannosidase. A composition for use in the processes as described herein may also comprise one or more pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase. In addition, one or more of the following enzymes, an amylase, a protease, a lipase, a ligninase, a hexosyltransferase, a glucuronidase, an expansin, a cellulose induced protein or a cellulose integrating protein or like protein may be present in a composition for use in the processes as described herein (these are referred to as auxiliary activities above).

As used herein, lytic polysaccharide monooxygenases are enzymes that have recently been classified by CAZy in family AA9 (Auxiliary Activity Family 9) or family AA10 (Auxiliary Activity Family 10). Ergo, there exist AA9 lytic polysaccharide monooxygenases and AA10 lytic polysaccharide monooxygenases. Lytic polysaccharide monooxygenases are able to open a crystalline glucan structure and enhance the action of cellulases on lignocellulose substrates. They are enzymes having cellulolytic enhancing activity. Lytic polysaccharide monooxygenases may also affect cello-oligosaccharides. According to the latest literature, (see Isaksen et al., Journal of Biological Chemistry, vol. 289, no. 5, p. 2632-2642), proteins named GH61 (glycoside hydrolase family 61 or sometimes referred to EGIV) are lytic polysaccharide monooxygenases. GH61 was originally classified as endoglucanase based on measurement of very weak endo-1,4-β-d-glucanase activity in one family member but have recently been reclassified by CAZy in family AA9. CBM33 (family 33 carbohydrate-binding module) is also a lytic polysaccharide monooxygenase (see Isaksen et al, Journal of Biological Chemistry, vol. 289, no. 5, pp. 2632-2642). CAZy has recently reclassified CBM33 in the AA10 family.

In an embodiment the lytic polysaccharide monooxygenase comprises an AA9 lytic polysaccharide monooxygenase. This means that at least one of the lytic polysaccharide monooxygenases in the enzyme composition is an AA9 lytic polysaccharide monooxygenase. In an embodiment, all lytic polysaccharide monooxygenases in the enzyme composition are AA9 lytic polysaccharide monooxygenase.

In an embodiment the enzyme composition comprises a lytic polysaccharide monooxygenase from Thermoascus, such as Thermoascus aurantiacus, such as the one described in WO 2005/074656 as SEQ ID NO:2 and SEQ ID NO:1 in WO2014/130812 and in WO 2010/065830; or from Thielavia, such as Thielavia terrestris, such as the one described in WO 2005/074647 as SEQ ID NO: 8 or SEQ ID NO:4 in WO2014/130812 and in WO 2008/148131, and WO 2011/035027; or from Aspergillus, such as Aspergillus fumigatus, such as the one described in WO 2010/138754 as SEQ ID NO:2 or SEQ ID NO: 3 in WO2014/130812; or from Penicillium, such as Penicillium emersonii, such as the one disclosed as SEQ ID NO:2 in WO 2011/041397 or SEQ ID NO:2 in WO2014/130812. Other suitable lytic polysaccharide monooxygenases include, but are not limited to, Trichoderma reesei (see WO 2007/089290), Myceliophthora thermophila (see WO 2009/085935, WO 2009/085859, WO 2009/085864, WO 2009/085868), Penicillium pinophilum (see WO 2011/005867), Thermoascus sp. (see WO 2011/039319), and Thermoascus crustaceous (see WO 2011/041504). Other cellulolytic enzymes that may be comprised in the enzyme composition are described in WO 98/13465, WO 98/015619, WO 98/015633, WO 99/06574, WO 99/10481, WO 99/025847, WO 99/031255, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. Nos. 5,457,046, 5,648,263, and 5,686,593, to name just a few. In a preferred embodiment, the lytic polysaccharide monooxygenase is from Rasamsonia, e.g. Rasamsonia emersonii (see WO 2012/000892).

As used herein, endoglucanases are enzymes which are capable of catalyzing the endohydrolysis of 1,4-β-D-glucosidic linkages in cellulose, lichenin or cereal β-D-glucans. They belong to EC 3.2.1.4 and may also be capable of hydrolyzing 1,4-linkages in β-D-glucans also containing 1,3-linkages. Endoglucanases may also be referred to as cellulases, avicelases, endoglucan hydrolases, β-1,4-glucanases, carboxymethyl cellulases, celludextrinases, endo-1,4-β-D-glucanases, endo-1,4-β-D-glucanohydrolases or endo-1,4-β-glucanases.

In an embodiment the endoglucanase comprises a GH5 endoglucanase and/or a GH7 endoglucanase. This means that at least one of the endoglucanases in the enzyme composition is a GH5 endoglucanase or a GH7 endoglucanase. In case there are more endoglucanases in the enzyme composition, these endoglucanases can be GH5 endoglucanases, GH7 endoglucanases or a combination of GH5 endoglucanases and GH7 endoglucanases. In a preferred embodiment the endoglucanase comprises a GH5 endoglucanase.

In an embodiment the enzyme composition comprises an endoglucanase from Trichoderma, such as Trichoderma reesei; from Humicola, such as a strain of Humicola insolens; from Aspergillus, such as Aspergillus aculeatus or Aspergillus kawachii; from Erwinia, such as Erwinia carotovara; from Fusarium, such as Fusarium oxysporum; from Thielavia, such as Thielavia terrestris; from Humicola, such as Humicola grisea var. thermoidea or Humicola insolens; from Melanocarpus, such as Melanocarpus albomyces; from Neurospora, such as Neurospora crassa; from Myceliophthora, such as Myceliophthora thermophila; from Cladorrhinum, such as Cladorrhinum foecundissimum; and/or from Chrysosporium, such as a strain of Chrysosporium lucknowense. In a preferred embodiment the endoglucanase is from Rasamsonia, such as a strain of Rasamsonia emersonii (see WO 01/70998). In an embodiment even a bacterial endoglucanase can be used including, but are not limited to, Acidothermus cellulolyticus endoglucanase (see WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (see WO 05/093050); and Thermobifida fusca endoglucanase V (see WO 05/093050).

As used herein, beta-xylosidases (EC 3.2.1.37) are polypeptides which are capable of catalysing the hydrolysis of 1,4-β-D-xylans, to remove successive D-xylose residues from the non-reducing termini. Beta-xylosidases may also hydrolyze xylobiose. Beta-xylosidase may also be referred to as xylan 1,4-β-xylosidase, 1,4-β-D-xylan xylohydrolase, exo-1,4-β-xylosidase or xylobiase.

In an embodiment the beta-xylosidase comprises a GH3 beta-xylosidase. This means that at least one of the beta-xylosidases in the enzyme composition is a GH3 beta-xylosidase. In an embodiment all beta-xylosidases in the enzyme composition are GH3 beta-xylosidases.

In an embodiment the enzyme composition comprises a beta-xylosidase from Neurospora crassa, Aspergillus fumigatus or Trichoderma reesei. In a preferred embodiment the enzyme composition comprises a beta-xylosidase from Rasamsonia, such as Rasamsonia emersonii (see WO 2014/118360).

As used herein, an endoxylanase (EC 3.2.1.8) is any polypeptide which is capable of catalysing the endohydrolysis of 1,4-β-D-xylosidic linkages in xylans. This enzyme may also be referred to as endo-1,4-β-xylanase or 1,4-β-D-xylan xylanohydrolase. An alternative is EC 3.2.1.136, a glucuronoarabinoxylan endoxylanase, an enzyme that is able to hydrolyze 1,4 xylosidic linkages in glucuronoarabinoxylans.

In an embodiment the endoxylanase comprises a GH10 xylanase. This means that at least one of the endoxylanases in the enzyme composition is a GH10 xylanase. In an embodiment all endoxylanases in the enzyme composition are GH10 xylanases.

In an embodiment the enzyme composition comprises an endoxylanase from Aspergillus aculeatus (see WO 94/21785), Aspergillus fumigatus (see WO 2006/078256), Penicillium pinophilum (see WO 2011/041405), Penicillium sp. (see WO 2010/126772), Thielavia terrestris NRRL 8126 (see WO 2009/079210), Talaromyces leycettanus, Thermobifida fusca, or Trichophaea saccata GH10 (see WO 2011/057083). In a preferred embodiment the enzyme composition comprises an endoxylanase from Rasamsonia, such as Rasamsonia emersonii (see WO 02/24926).

As used herein, a beta-glucosidase (EC 3.2.1.21) is any polypeptide which is capable of catalysing the hydrolysis of terminal, non-reducing β-D-glucose residues with release of β-D-glucose. Such a polypeptide may have a wide specificity for β-D-glucosides and may also hydrolyze one or more of the following: a β-D-galactoside, an α-L-arabinoside, a β-D-xyloside or a β-D-fucoside. This enzyme may also be referred to as amygdalase, β-D-glucoside glucohydrolase, cellobiase or gentobiase.

In an embodiment the enzyme composition comprises a beta-glucosidase from Aspergillus, such as Aspergillus oryzae, such as the one disclosed in WO 02/095014 or the fusion protein having beta-glucosidase activity disclosed in WO 2008/057637, or Aspergillus fumigatus, such as the one disclosed as SEQ ID NO:2 in WO 2005/047499 or SEQ ID NO:5 in WO 2014/130812 or an Aspergillus fumigatus beta-glucosidase variant, such as one disclosed in WO 2012/044915, such as one with the following substitutions: F100D, S283G, N456E, F512Y (using SEQ ID NO: 5 in WO 2014/130812 for numbering), or Aspergillus aculeatus, Aspergillus niger or Aspergillus kawachi. In another embodiment the beta-glucosidase is derived from Penicillium, such as Penicillium brasilianum disclosed as SEQ ID NO:2 in WO 2007/019442, or from Trichoderma, such as Trichoderma reesei, such as ones described in U.S. Pat. Nos. 6,022,725, 6,982,159, 7,045,332, 7,005,289, US 2006/0258554 US 2004/0102619. In an embodiment, even a bacterial beta-glucosidase can be used. In another embodiment the beta-glucosidase is derived from Thielavia terrestris (WO 2011/035029) or Trichophaea saccata (WO 2007/019442). In a preferred embodiment the enzyme composition comprises a beta-glucosidase from Rasamsonia, such as Rasamsonia emersonii (see WO 2012/000886).

As used herein, a cellobiohydrolase (EC 3.2.1.91) is any polypeptide which is capable of catalyzing the hydrolysis of 1,4-β-D-glucosidic linkages in cellulose or cellotetraose, releasing cellobiose from the ends of the chains. This enzyme may also be referred to as cellulase 1,4-β-cellobiosidase, 1,4-β-cellobiohydrolase, 1,4-β-D-glucan cellobiohydrolase, avicelase, exo-1,4-β-D-glucanase, exocellobiohydrolase or exoglucanase.

In an embodiment the enzyme composition comprises a cellobiohydrolase I from Aspergillus, such as Aspergillus fumigatus, such as the Cel7A CBH I disclosed in SEQ ID NO:6 in WO 2011/057140 or SEQ ID NO:6 in WO 2014/130812; from Trichoderma, such as Trichoderma reesei; from Chaetomium, such as Chaetomium thermophilum; from Talaromyces, such as Talaromyces leycettanus or from Penicillium, such as Penicillium emersonii. In a preferred embodiment the enzyme composition comprises a cellobiohydrolase I from Rasamsonia, such as Rasamsonia emersonii (see WO 2010/122141).

In an embodiment the enzyme composition comprises a cellobiohydrolase II from Aspergillus, such as Aspergillus fumigatus, such as the one in SEQ ID NO:7 in WO 2014/130812 or from Trichoderma, such as Trichoderma reesei, or from Talaromyces, such as Talaromyces leycettanus, or from Thielavia, such as Thielavia terrestris, such as cellobiohydrolase II CEL6A from Thielavia terrestris. In a preferred embodiment the enzyme composition comprises a cellobiohydrolase II from Rasamsonia, such as Rasamsonia emersonii (see WO 2011/098580).

In an embodiment the enzyme composition also comprises one or more of the below mentioned enzymes.

As used herein, a β-(1,3)(1,4)-glucanase (EC 3.2.1.73) is any polypeptide which is capable of catalysing the hydrolysis of 1,4-β-D-glucosidic linkages in β-D-glucans containing 1,3- and 1,4-bonds. Such a polypeptide may act on lichenin and cereal β-D-glucans, but not on β-D-glucans containing only 1,3- or 1,4-bonds. This enzyme may also be referred to as licheninase, 1,3-1,4-β-D-glucan 4-glucanohydrolase, β-glucanase, endo-β-1,3-1,4 glucanase, lichenase or mixed linkage β-glucanase. An alternative for this type of enzyme is EC 3.2.1.6, which is described as endo-1,3(4)-beta-glucanase. This type of enzyme hydrolyses 1,3- or 1,4-linkages in beta-D-glucanse when the glucose residue whose reducing group is involved in the linkage to be hydrolysed is itself substituted at C-3. Alternative names include endo-1,3-beta-glucanase, laminarinase, 1,3-(1,3;1,4)-beta-D-glucan 3 (4) glucanohydrolase. Substrates include laminarin, lichenin and cereal beta-D-glucans.

As used herein, an α-L-arabinofuranosidase (EC 3.2.1.55) is any polypeptide which is capable of acting on α-L-arabinofuranosides, α-L-arabinans containing (1,2) and/or (1,3)- and/or (1,5)-linkages, arabinoxylans and arabinogalactans. This enzyme may also be referred to as α-N-arabinofuranosidase, arabinofuranosidase or arabinosidase. Examples of arabinofuranosidases that may be comprised in the enzyme composition include, but are not limited to, arabinofuranosidases from Aspergillus niger, Humicola insolens DSM 1800 (see WO 2006/114094 and WO 2009/073383) and M. giganteus (see WO 2006/114094).

As used herein, an α-D-glucuronidase (EC 3.2.1.139) is any polypeptide which is capable of catalysing a reaction of the following form: alpha-D-glucuronoside+H(2)O=an alcohol+D-glucuronate. This enzyme may also be referred to as alpha-glucuronidase or alpha-glucosiduronase. These enzymes may also hydrolyse 4-O-methylated glucoronic acid, which can also be present as a substituent in xylans. An alternative is EC 3.2.1.131: xylan alpha-1,2-glucuronosidase, which catalyses the hydrolysis of alpha-1,2-(4-O-methyl)glucuronosyl links. Examples of alpha-glucuronidases that may be comprised in the enzyme composition include, but are not limited to, alpha-glucuronidases from Aspergillus clavatus, Aspergillus fumigatus, Aspergillus niger, Aspergillus terreus, Humicola insolens (see WO 2010/014706), Penicillium aurantiogriseum (see WO 2009/068565) and Trichoderma reesei.

As used herein, an acetyl xylan esterase (EC 3.1.1.72) is any polypeptide which is capable of catalysing the deacetylation of xylans and xylo-oligosaccharides. Such a polypeptide may catalyze the hydrolysis of acetyl groups from polymeric xylan, acetylated xylose, acetylated glucose, alpha-napthyl acetate or p-nitrophenyl acetate but, typically, not from triacetylglycerol. Such a polypeptide typically does not act on acetylated mannan or pectin. Examples of acetylxylan esterases that may be comprised in the enzyme composition include, but are not limited to, acetylxylan esterases from Aspergillus aculeatus (see WO 2010/108918), Chaetomium globosum, Chaetomium gracile, Humicola insolens DSM 1800 (see WO 2009/073709), Hypocrea jecorina (see WO 2005/001036), Myceliophtera thermophila (see WO 2010/014880), Neurospora crassa, Phaeosphaeria nodorum and Thielavia terrestris NRRL 8126 (see WO 2009/042846). In a preferred embodiment the enzyme composition comprises an acetyl xylan esterase from Rasamsonia, such as Rasamsonia emersonii (see WO 2010/000888)

As used herein, a feruloyl esterase (EC 3.1.1.73) is any polypeptide which is capable of catalysing a reaction of the form: feruloyl-saccharide+H₂O=ferulate+saccharide. The saccharide may be, for example, an oligosaccharide or a polysaccharide. It may typically catalyse the hydrolysis of the 4-hydroxy-3-methoxycinnamoyl (feruloyl) group from an esterified sugar, which is usually arabinose in ‘natural’ substrates. p-nitrophenol acetate and methyl ferulate are typically poorer substrates. This enzyme may also be referred to as cinnamoyl ester hydrolase, ferulic acid esterase or hydroxycinnamoyl esterase. It may also be referred to as a hemicellulase accessory enzyme, since it may help xylanases and pectinases to break down plant cell wall hemicellulose and pectin. Examples of feruloyl esterases (ferulic acid esterases) that may be comprised in the enzyme composition include, but are not limited to, feruloyl esterases form Humicola insolens DSM 1800 (see WO 2009/076122), Neosartorya fischeri, Neurospora crassa, Penicillium aurantiogriseum (see WO 2009/127729), and Thielavia terrestris (see WO 2010/053838 and WO 2010/065448).

As used herein, a coumaroyl esterase (EC 3.1.1.73) is any polypeptide which is capable of catalysing a reaction of the form: coumaroyl-saccharide+H(2)O=coumarate+saccharide. The saccharide may be, for example, an oligosaccharide or a polysaccharide. This enzyme may also be referred to as trans-4-coumaroyl esterase, trans-p-coumaroyl esterase, p-coumaroyl esterase or p-coumaric acid esterase. This enzyme also falls within EC 3.1.1.73 so may also be referred to as a feruloyl esterase.

As used herein, an α-galactosidase (EC 3.2.1.22) is any polypeptide which is capable of catalysing the hydrolysis of terminal, non-reducing α-D-galactose residues in α-D-galactosides, including galactose oligosaccharides, galactomannans, galactans and arabinogalactans. Such a polypeptide may also be capable of hydrolyzing α-D-fucosides. This enzyme may also be referred to as melibiase.

As used herein, a β-galactosidase (EC 3.2.1.23) is any polypeptide which is capable of catalysing the hydrolysis of terminal non-reducing β-D-galactose residues in β-D-galactosides. Such a polypeptide may also be capable of hydrolyzing α-L-arabinosides. This enzyme may also be referred to as exo-(1->4)-β-D-galactanase or lactase.

As used herein, a β-mannanase (EC 3.2.1.78) is any polypeptide which is capable of catalysing the random hydrolysis of 1,4-β-D-mannosidic linkages in mannans, galactomannans and glucomannans. This enzyme may also be referred to as mannan endo-1,4-β-mannosidase or endo-1,4-mannanase.

As used herein, a β-mannosidase (EC 3.2.1.25) is any polypeptide which is capable of catalysing the hydrolysis of terminal, non-reducing β-D-mannose residues in β-D-mannosides. This enzyme may also be referred to as mannanase or mannase.

As used herein, an endo-polygalacturonase (EC 3.2.1.15) is any polypeptide which is capable of catalysing the random hydrolysis of 1,4-α-D-galactosiduronic linkages in pectate and other galacturonans. This enzyme may also be referred to as polygalacturonase pectin depolymerase, pectinase, endopolygalacturonase, pectolase, pectin hydrolase, pectin polygalacturonase, poly-α-1,4-galacturonide glycanohydrolase, endogalacturonase; endo-D-galacturonase or poly(1,4-α-D-galacturonide) glycanohydrolase.

As used herein, a pectin methyl esterase (EC 3.1.1.11) is any enzyme which is capable of catalysing the reaction: pectin+n H₂O=n methanol+pectate. The enzyme may also be known as pectinesterase, pectin demethoxylase, pectin methoxylase, pectin methylesterase, pectase, pectinoesterase or pectin pectylhydrolase.

As used herein, an endo-galactanase (EC 3.2.1.89) is any enzyme capable of catalysing the endohydrolysis of 1,4-β-D-galactosidic linkages in arabinogalactans. The enzyme may also be known as arabinogalactan endo-1,4-β-galactosidase, endo-1,4-β-galactanase, galactanase, arabinogalactanase or arabinogalactan 4-β-D-galactanohydrolase.

As used herein, a pectin acetyl esterase is defined herein as any enzyme which has an acetyl esterase activity which catalyses the deacetylation of the acetyl groups at the hydroxyl groups of GalUA residues of pectin.

As used herein, an endo-pectin lyase (EC 4.2.2.10) is any enzyme capable of catalysing the eliminative cleavage of (1→4)-α-D-galacturonan methyl ester to give oligosaccharides with 4-deoxy-6-O-methyl-α-D-galact-4-enuronosyl groups at their non-reducing ends. The enzyme may also be known as pectin lyase, pectin trans-eliminase; endo-pectin lyase, polymethylgalacturonic transeliminase, pectin methyltranseliminase, pectolyase, PL, PNL or PMGL or (1→4)-6-O-methyl-α-D-galacturonan lyase.

As used herein, a pectate lyase (EC 4.2.2.2) is any enzyme capable of catalysing the eliminative cleavage of (1→4)-α-D-galacturonan to give oligosaccharides with 4-deoxy-α-D-galact-4-enuronosyl groups at their non-reducing ends. The enzyme may also be known polygalacturonic transeliminase, pectic acid transeliminase, polygalacturonate lyase, endopectin methyltranseliminase, pectate transeliminase, endogalacturonate transeliminase, pectic acid lyase, pectic lyase, α-1,4-D-endopolygalacturonic acid lyase, PGA lyase, PPase-N, endo-α-1,4-polygalacturonic acid lyase, polygalacturonic acid lyase, pectin trans-eliminase, polygalacturonic acid trans-eliminase or (1→4)-α-D-galacturonan lyase.

As used herein, an alpha rhamnosidase (EC 3.2.1.40) is any polypeptide which is capable of catalysing the hydrolysis of terminal non-reducing α-L-rhamnose residues in α-L-rhamnosides or alternatively in rhamnogalacturonan. This enzyme may also be known as α-L-rhamnosidase T, α-L-rhamnosidase N or α-L-rhamnoside rhamnohydrolase.

As used herein, exo-galacturonase (EC 3.2.1.82) is any polypeptide capable of hydrolysis of pectic acid from the non-reducing end, releasing digalacturonate. The enzyme may also be known as exo-poly-α-galacturonosidase, exopolygalacturonosidase or exopolygalacturanosidase.

As used herein, exo-galacturonase (EC 3.2.1.67) is any polypeptide capable of catalysing: (1,4-α-D-galacturonide)ₙ+H₂O=(1,4-α-D-galacturonide)ₙ₋₁+D-galacturonate. The enzyme may also be known as galacturan 1,4-α-galacturonidase, exopolygalacturonase, poly(galacturonate) hydrolase, exo-D-galacturonase, exo-D-galacturonanase, exopoly-D-galacturonase or poly(1,4-α-D-galacturonide) galacturonohydrolase.

As used herein, exopolygalacturonate lyase (EC 4.2.2.9) is any polypeptide capable of catalysing eliminative cleavage of 4-(4-deoxy-α-D-galact-4-enuronosyl)-D-galacturonate from the reducing end of pectate, i.e. de-esterified pectin. This enzyme may be known as pectate disaccharide-lyase, pectate exo-lyase, exopectic acid transeliminase, exopectate lyase, exopolygalacturonic acid-trans-eliminase, PATE, exo-PATE, exo-PGL or (1→4)-α-D-galacturonan reducing-end-disaccharide-lyase.

As used herein, rhamnogalacturonan hydrolase is any polypeptide which is capable of hydrolyzing the linkage between galactosyluronic acid and rhamnopyranosyl in an endo-fashion in strictly alternating rhamnogalacturonan structures, consisting of the disaccharide [(1,2-alpha-L-rhamnoyl-(1,4)-alpha-galactosyluronic acid].

As used herein, rhamnogalacturonan lyase is any polypeptide which is any polypeptide which is capable of cleaving α-L-Rhap-(1→4)-α-D-GalpA linkages in an endo-fashion in rhamnogalacturonan by beta-elimination.

As used herein, rhamnogalacturonan acetyl esterase is any polypeptide which catalyzes the deacetylation of the backbone of alternating rhamnose and galacturonic acid residues in rhamnogalacturonan.

As used herein, rhamnogalacturonan galacturonohydrolase is any polypeptide which is capable of hydrolyzing galacturonic acid from the non-reducing end of strictly alternating rhamnogalacturonan structures in an exo-fashion.

As used herein, xylogalacturonase is any polypeptide which acts on xylogalacturonan by cleaving the β-xylose substituted galacturonic acid backbone in an endo-manner. This enzyme may also be known as xylogalacturonan hydrolase.

As used herein, an α-L-arabinofuranosidase (EC 3.2.1.55) is any polypeptide which is capable of acting on α-L-arabinofuranosides, α-L-arabinans containing (1,2) and/or (1,3)- and/or (1,5)-linkages, arabinoxylans and arabinogalactans. This enzyme may also be referred to as α-N-arabinofuranosidase, arabinofuranosidase or arabinosidase.

As used herein, endo-arabinanase (EC 3.2.1.99) is any polypeptide which is capable of catalysing endohydrolysis of 1,5-α-arabinofuranosidic linkages in 1,5-arabinans. The enzyme may also be known as endo-arabinase, arabinan endo-1,5-α-L-arabinosidase, endo-1,5-α-L-arabinanase, endo-α-1,5-arabanase; endo-arabanase or 1,5-α-L-arabinan 1,5-α-L-arabinanohydrolase.

“Protease” includes enzymes that hydrolyze peptide bonds (peptidases), as well as enzymes that hydrolyze bonds between peptides and other moieties, such as sugars (glycopeptidases). Many proteases are characterized under EC 3.4 and are suitable for use in the processes as described herein. Some specific types of proteases include, cysteine proteases including pepsin, papain and serine proteases including chymotrypsins, carboxypeptidases and metalloendopeptidases.

“Lipase” includes enzymes that hydrolyze lipids, fatty acids, and acylglycerides, including phospoglycerides, lipoproteins, diacylglycerols, and the like. In plants, lipids are used as structural components to limit water loss and pathogen infection. These lipids include waxes derived from fatty acids, as well as cutin and suberin.

“Ligninase” includes enzymes that can hydrolyze or break down the structure of lignin polymers. Enzymes that can break down lignin include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic sugars (notably arabinose) and lignin. Ligninases include but are not limited to the following group of enzymes: lignin peroxidases (EC 1.11.1.14), manganese peroxidases (EC 1.11.1.13), laccases (EC 1.10.3.2) and feruloyl esterases (EC 3.1.1.73).

“Hexosyltransferase” (2.4.1-) includes enzymes which are capable of catalysing a transferase reaction, but which can also catalyze a hydrolysis reaction, for example of cellulose and/or cellulose degradation products. An example of a hexosyltransferase which may be used is a β-glucanosyltransferase. Such an enzyme may be able to catalyze degradation of (1,3)(1,4)glucan and/or cellulose and/or a cellulose degradation product.

“Glucuronidase” includes enzymes that catalyze the hydrolysis of a glucuronoside, for example β-glucuronoside to yield an alcohol. Many glucuronidases have been characterized and may be suitable for use, for example β-glucuronidase (EC 3.2.1.31), hyalurono-glucuronidase (EC 3.2.1.36), glucuronosyl-disulfoglucosamine glucuronidase (3.2.1.56), glycyrrhizinate β-glucuronidase (3.2.1.128) or α-D-glucuronidase (EC 3.2.1.139).

Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. As described herein, an expansin-like protein or swollenin-like protein may comprise one or both of such domains and/or may disrupt the structure of cell walls (such as disrupting cellulose structure), optionally without producing detectable amounts of reducing sugars.

A cellulose induced protein, for example the polypeptide product of the cip1 or cip2 gene or similar genes (see Foreman et al., J. Biol. Chem. 278(34), 31988-31997, 2003), a cellulose/cellulosome integrating protein, for example the polypeptide product of the cipA or cipC gene, or a scaffoldin or a scaffoldin-like protein. Scaffoldins and cellulose integrating proteins are multi-functional integrating subunits which may organize cellulolytic subunits into a multi-enzyme complex. This is accomplished by the interaction of two complementary classes of domain, i.e. a cohesion domain on scaffoldin and a dockerin domain on each enzymatic unit. The scaffoldin subunit also bears a cellulose-binding module (CBM) that mediates attachment of the cellulosome to its substrate. A scaffoldin or cellulose integrating protein may comprise one or both of such domains.

A catalase; the term “catalase” means a hydrogen-peroxide:hydrogen-peroxide oxidoreductase (EC 1.11.1.6 or EC 1.11.1.21) that catalyzes the conversion of two hydrogen peroxides to oxygen and two waters. Catalase activity can be determined by monitoring the degradation of hydrogen peroxide at 240 nm based on the following reaction: 2H₂O₂→2H₂O+O₂. The reaction is conducted in 50 mM phosphate pH 7.0 at 25° C. with 10.3 mM substrate (H₂O₂) and approximately 100 units of enzyme per ml. Absorbance is monitored spectrophotometrically within 16-24 seconds, which should correspond to an absorbance reduction from 0.45 to 0.4. One catalase activity unit can be expressed as one micromole of H₂O₂ degraded per minute at pH 7.0 and 25° C.

The term “amylase” as used herein means enzymes that hydrolyze alpha-1,4-glucosidic linkages in starch, both in amylose and amylopectin, such as alpha-amylase (EC 3.2.1.1), beta-amylase (EC 3.2.1.2), glucan 1,4-alpha-glucosidase (EC 3.2.1.3), glucan 1,4-alpha-maltotetraohydrolase (EC 3.2.1.60), glucan 1,4-alpha-maltohexaosidase (EC 3.2.1.98), glucan 1,4-alpha-maltotriohydrolase (EC 3.2.1.116) and glucan 1,4-alpha-maltohydrolase (EC 3.2.1.133), and enzymes that hydrolyze alpha-1,6-glucosidic linkages, being the branch-points in amylopectin, such as pullulanase (EC 3.2.1.41) and limit dextinase (EC 3.2.1.142).

A composition for use in the processes as described herein may be composed of enzymes from (1) commercial suppliers; (2) cloned genes expressing enzymes; (3) broth (such as that resulting from growth of a microbial strain in media, wherein the strains secrete proteins and enzymes into the media; (4) cell lysates of strains grown as in (3); and/or (5) plant material expressing enzymes. Different enzymes in a composition of the invention may be obtained from different sources.

The enzymes can be produced either exogenously in microorganisms, yeasts, fungi, bacteria or plants, then isolated and added, for example, to lignocellulosic material. Alternatively, the enzyme may be produced in a fermentation that uses (pretreated) lignocellulosic material (such as corn stover or wheat straw) to provide nutrition to an organism that produces an enzyme(s). In this manner, plants that produce the enzymes may themselves serve as a lignocellulosic material and be added into lignocellulosic material.

In the uses and processes described herein, the components of the compositions described above may be provided concomitantly (i.e. as a single composition per se) or separately or sequentially.

In an embodiment the enzyme composition comprises a whole fermentation broth of a fungus, preferably a whole fermentation broth of a filamentous fungus, more preferably a whole fermentation broth of Rasamsonia. The whole fermentation broth can be prepared from fermentation of non-recombinant and/or recombinant filamentous fungi. In an embodiment the filamentous fungus is a recombinant filamentous fungus comprising one or more genes which can be homologous or heterologous to the filamentous fungus. In an embodiment, the filamentous fungus is a recombinant filamentous fungus comprising one or more genes which can be homologous or heterologous to the filamentous fungus wherein the one or more genes encode enzymes that can degrade a cellulosic substrate. The whole fermentation broth may comprise any of the polypeptides described above or any combination thereof.

Preferably, the enzyme composition is a whole fermentation broth wherein the cells are killed. The whole fermentation broth may contain organic acid(s) (used for killing the cells), killed cells and/or cell debris, and culture medium.

Generally, filamentous fungi are cultivated in a cell culture medium suitable for production of enzymes capable of hydrolyzing a cellulosic substrate. The cultivation takes place in a suitable nutrient medium comprising carbon and nitrogen sources and inorganic salts, using procedures known in the art. Suitable culture media, temperature ranges and other conditions suitable for growth and cellulase and/or hemicellulase and/or pectinase production are known in the art. The whole fermentation broth can be prepared by growing the filamentous fungi to stationary phase and maintaining the filamentous fungi under limiting carbon conditions for a period of time sufficient to express the one or more cellulases and/or hemicellulases and/or pectinases. Once enzymes, such as cellulases and/or hemicellulases and/or pectinases, are secreted by the filamentous fungi into the fermentation medium, the whole fermentation broth can be used. The whole fermentation broth of the present invention may comprise filamentous fungi. In some embodiments, the whole fermentation broth comprises the unfractionated contents of the fermentation materials derived at the end of the fermentation. Typically, the whole fermentation broth comprises the spent culture medium and cell debris present after the filamentous fungi is grown to saturation, incubated under carbon-limiting conditions to allow protein synthesis (particularly, expression of cellulases and/or hemicellulases and/or pectinases). In some embodiments, the whole fermentation broth comprises the spent cell culture medium, extracellular enzymes and filamentous fungi. In some embodiments, the filamentous fungi present in whole fermentation broth can be lysed, permeabilized, or killed using methods known in the art to produce a cell-killed whole fermentation broth. In an embodiment, the whole fermentation broth is a cell-killed whole fermentation broth, wherein the whole fermentation broth containing the filamentous fungi cells are lysed or killed. In some embodiments, the cells are killed by lysing the filamentous fungi by chemical and/or pH treatment to generate the cell-killed whole broth of a fermentation of the filamentous fungi. In some embodiments, the cells are killed by lysing the filamentous fungi by chemical and/or pH treatment and adjusting the pH of the cell-killed fermentation mix to a suitable pH. In an embodiment, the whole fermentation broth comprises a first organic acid component comprising at least one 1-5 carbon organic acid and/or a salt thereof and a second organic acid component comprising at least 6 or more carbon organic acid and/or a salt thereof. In an embodiment, the first organic acid component is acetic acid, formic acid, propionic acid, a salt thereof, or any combination thereof and the second organic acid component is benzoic acid, cyclohexanecarboxylic acid, 4-methylvaleric acid, phenylacetic acid, a salt thereof, or any combination thereof.

The term “whole fermentation broth” as used herein refers to a preparation produced by cellular fermentation that undergoes no or minimal recovery and/or purification. For example, whole fermentation broths are produced when microbial cultures are grown to saturation, incubated under carbon-limiting conditions to allow protein synthesis (e.g., expression of enzymes by host cells) and secretion into cell culture medium. Typically, the whole fermentation broth is unfractionated and comprises spent cell culture medium, extracellular enzymes, and microbial, preferably non-viable, cells.

If needed, the whole fermentation broth can be fractionated and the one or more of the fractionated contents can be used. For instance, the killed cells and/or cell debris can be removed from a whole fermentation broth to provide a composition that is free of these components.

The whole fermentation broth may further comprise a preservative and/or anti-microbial agent. Such preservatives and/or agents are known in the art.

The whole fermentation broth as described herein is typically a liquid, but may contain insoluble components, such as killed cells, cell debris, culture media components, and/or insoluble enzyme(s). In some embodiments, insoluble components may be removed to provide a clarified whole fermentation broth.

In an embodiment, the whole fermentation broth may be supplemented with one or more enzyme activities that are not expressed endogenously or expressed at relatively low level by the filamentous fungi, to improve the degradation of the cellulosic substrate, for example, to fermentable sugars such as glucose or xylose. The supplemental enzyme(s) can be added as a supplement to the whole fermentation broth and the enzymes may be a component of a separate whole fermentation broth, or may be purified, or minimally recovered and/or purified.

In an embodiment, the whole fermentation broth comprises a whole fermentation broth of a fermentation of a recombinant filamentous fungus overexpressing one or more enzymes to improve the degradation of the cellulosic substrate. Alternatively, the whole fermentation broth can comprise a mixture of a whole fermentation broth of a fermentation of a non-recombinant filamentous fungus and a recombinant filamentous fungus overexpressing one or more enzymes to improve the degradation of the cellulosic substrate. In an embodiment, the whole fermentation broth comprises a whole fermentation broth of a fermentation of a filamentous fungus overexpressing beta-glucosidase or endoglucanase. Alternatively, the whole fermentation broth for use in the present methods and reactive compositions can comprise a mixture of a whole fermentation broth of a fermentation of a non-recombinant filamentous fungus and a whole fermentation broth of a fermentation of a recombinant filamentous fungus overexpressing a beta-glucosidase or endoglucanase.

Cellulosic material as used herein includes any cellulose containing material. Preferably, cellulosic material as used herein includes lignocellulosic and/or hemicellulosic material. Most preferably cellulosic material as used herein is lignocellulosic material. Cellulosic material suitable for use in the processes as described herein includes biomass, e.g. virgin biomass and/or non-virgin biomass such as agricultural biomass, commercial organics, construction and demolition debris, municipal solid waste, waste paper and yard waste. Common forms of biomass include trees, shrubs and grasses, wheat, rye, oat, wheat straw, sugar cane, cane straw, sugar cane bagasse, switch grass, miscanthus, energy cane, cassava, molasse, barley, corn, corn stover, corn fiber, corn husks, corn cobs, canola stems, soybean stems, sweet sorghum, corn kernel including fiber from kernels, distillers dried grains (DDGS), products and by-products from milling of grains such as corn, wheat and barley (including wet milling and dry milling) often called “bran or fibre” as well as municipal solid waste, waste paper and yard waste. The biomass can also be, but is not limited to, herbaceous material, agricultural residues, forestry residues, municipal solid wastes, waste woods (type A, B and/or C), waste paper, and pulp and paper mill residues. “Agricultural biomass” includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, fruit peels, vines, sugar beet, sugar beet pulp, wheat midlings, oat hulls, and hard and soft woods (not including woods with deleterious materials). In addition, agricultural biomass includes organic waste materials generated from agricultural processes including farming and forestry activities, specifically including forestry wood waste. Agricultural biomass may be any of the afore-mentioned singularly or in any combination or mixture thereof.

The cellulosic material is pretreated before the hydrolysis step. Pretreatment methods are known in the art and include, but are not limited to, heat, mechanical, chemical modification, biological modification and any combination thereof. Pretreatment is typically performed in order to enhance the accessibility of the cellulosic material to enzymatic hydrolysis and/or hydrolyse the hemicellulose and/or solubilize the hemicellulose and/or cellulose and/or lignin, in the cellulosic material. In an embodiment, the pretreatment comprises treating the cellulosic material with steam explosion, hot water treatment or treatment with dilute acid or dilute base. Examples of pretreatment methods include, but are not limited to, steam treatment (e.g. treatment at 100-260° C., at a pressure of 7-45 bar, at neutral pH, for 1-10 minutes), dilute acid treatment (e.g. treatment with 0.1-5% H₂SO₄ and/or SO₂ and/or HNO₃ and/or HCl, in presence or absence of steam, at 120-200° C., at a pressure of 2-15 bar, at acidic pH, for 2-30 minutes), organosolv treatment (e.g. treatment with 1-1.5% H₂SO₄ in presence of organic solvent and steam, at 160-200° C., at a pressure of 7-30 bar, at acidic pH, for 30-60 minutes), lime treatment (e.g. treatment with 0.1-2% NaOH/Ca(OH)₂ in the presence of water/steam at 60-160° C., at a pressure of 1-10 bar, at alkaline pH, for 60-4800 minutes), ARP treatment (e.g. treatment with 5-15% NH₃, at 150-180° C., at a pressure of 9-17 bar, at alkaline pH, for 10-90 minutes), AFEX treatment (e.g. treatment with >15% NH₃, at 60-140° C., at a pressure of 8-20 bar, at alkaline pH, for 5-30 minutes). In an embodiment the pretreatment is done in the absence of oxygen.

The cellulosic material may be washed. In an embodiment the cellulosic material may be washed after the pretreatment. The washing step may be used to remove water soluble compounds that may act as inhibitors for the fermentation and/or hydrolysis step. The washing step may be conducted in manner known to the skilled person. Next to washing, other detoxification methods do exist. The cellulosic material may also be detoxified by any (or any combination) of these methods which include, but are not limited to, solid/liquid separation, vacuum evaporation, extraction, adsorption, neutralization, overliming, addition of reducing agents, addition of detoxifying enzymes such as laccases or peroxidases, addition of microorganisms capable of detoxification of hydrolysates.

The enzyme composition as described herein can extremely effectively hydrolyze cellulosic material, for example corn stover, wheat straw, cane straw, and/or sugar cane bagasse, which can then be further converted into a product, such as an enzyme composition.

In an embodiment the enzyme composition is used in the enzymatic hydrolysis in an amount of 4.5 mg to 15 mg protein/gram dry matter weight of glucans in the cellulosic material. In an embodiment the enzyme composition is used in the enzymatic hydrolysis in an amount of 5 mg to 14 mg protein/gram dry matter weight of glucans in the cellulosic material. In an embodiment the enzyme composition is used in the enzymatic hydrolysis in an amount of 6 mg to 12 mg protein/gram dry matter weight of glucans in the cellulosic material.

Protein is measured according to TCA-Biuret analysis as described herein.

In an embodiment the dry matter content in the hydrolysis is from 10% to 40% (w/w) In an embodiment the pretreated cellulosic material that is hydrolysed has a dry matter content of 10 to 40% (w/w). In an embodiment the dry matter content of the cellulosic material in the enzymatic hydrolysis is from 10% to 40% (w/w), from 11% to 35% (w/w), from 12% to 30% (w/w), from 13% to 29% (w/w), from 14% to 28% (w/w), and preferably from 15% to 25% (w/w).

In an embodiment the hydrolysis step is conducted at a temperature of 40-90° C., preferably 45-70° C., more preferably 55-65° C.

In an embodiment the fermentation is done in a reactor. In an embodiment the fermentation may also be done in two, three, four, five, six, seven, eight, nine, ten or even more reactors. So, the term “reactor” is not limited to a single reactor but may mean multiple reactors.

In an embodiment the fermentation is done in a reactor having a volume of 1-5000 m³. In case multiple reactors are used in the fermentation of the processes as described herein, they may have the same volume, but also may have a different volume.

In an embodiment the reactor in which the fermentation is done has a ratio height to diameter of 2:1 to 8:1.

In an embodiment the fermentation is carried out by a fungus as already described above. The fungus ferments the hydrolysate to produce the enzyme composition.

The invention also pertains to a hydrolysate comprising 500-900 g sugars/kg dry matter hydrolysate and 0.5-3.5% (w/w) of a hydroxide of an alkali metal and/or a hydroxide of an alkaline earth metal. In an embodiment the hydrolysate comprises 500-900 g sugars/kg dry matter hydrolysate and 1.0-3.0% (w/w) of a hydroxide of an alkali metal and/or a hydroxide of an alkaline earth metal. In a preferred embodiment the hydrolysate comprises 500-900 g sugars/kg dry matter hydrolysate and 1.5-2.5% (w/w) of a hydroxide of an alkali metal and/or a hydroxide of an alkaline earth metal. Suitable hydroxides of an alkali metal and/or a hydroxides of an alkaline earth metal have been described herein.

The invention also pertains to a hydrolysate comprising 500-900 g sugars/kg dry matter hydrolysate and 0.5-3.5% (w/w) of a strong base. Suitable strong bases have been described herein. In an embodiment the hydrolysate comprises 500-900 g sugars/kg dry matter hydrolysate and 1.0-3.0% (w/w) of a strong base. In a preferred embodiment the hydrolysate comprises 500-900 g sugars/kg dry matter hydrolysate and 1.5-2.5% (w/w) of a strong base.

In a preferred embodiment the hydrolysate is prepared as described herein. In a preferred embodiment the hydrolysate is prepared by pretreating cellulosic material and enzymatically hydrolysing the pretreated cellulosic material to obtain the hydrolysate. The hydrolysate may be prepared by performing steps (a) and (b) of the processes as described herein.

The invention also pertains to a fermentation mixture comprising a hydrolysate, a fungus and 0.02-20 g of a hydroxide of an alkali metal and/or a hydroxide of an alkaline earth metal per kg fermentation mixture. In an embodiment the fermentation mixture comprises a hydrolysate, a fungus and 0.03-18 g of a hydroxide of an alkali metal and/or a hydroxide of an alkaline earth metal per kg fermentation mixture. In an embodiment the fermentation mixture comprises a hydrolysate, a fungus and 0.04-16 g of a hydroxide of an alkali metal and/or a hydroxide of an alkaline earth metal per kg fermentation mixture. In a preferred embodiment the fermentation mixture comprises a hydrolysate, a fungus and 0.05-15 g of a hydroxide of an alkali metal and/or a hydroxide of an alkaline earth metal per kg fermentation mixture. The hydrolysate may be the hydrolysate as described above. The hydrolysate may be prepared as described herein. The hydrolysate may be prepared by pretreating cellulosic material and enzymatically hydrolysing the pretreated cellulosic material to obtain the hydrolysate. The hydrolysate may be prepared by performing steps (a) and (b) of the processes as described herein. The fungus may be a fungus as described herein. The fermentation mixture may further comprise a cellulase, a hemicellulase and/or a pectinase. Suitable cellulases, hemicellulases and/or pectinases have been described herein. The fermentation mixture may be prepared as described herein. In an embodiment the fermentation mixture is prepared by pretreating cellulosic material, enzymatically hydrolysing the pretreated cellulosic material to obtain a hydrolysate and fermenting the hydrolysate

### EXAMPLES

### Example 1

### pH Control in Enzymatic Hydrolysis of Corn Stover

The effect of using different titrants to adjust and maintain the pH at a constant value during the enzymatic hydrolysis of pretreated carbohydrate material is shown in this example.

Rasamsonia emersonii cellulase cocktail (i.e. a whole fermentation broth) was produced according to the methods as described in WO 2011/000949. Moreover, Rasamsonia emersonii beta-glucosidase as described in WO2012/000890 was used in the experiments.

The protein concentration of the cellulase cocktail was determined using a biuret method. Cocktail samples were diluted on weight basis with water and centrifugated for 5 minutes at >14000×g. Bovine serum albumin (BSA) dilutions (0.5, 1, 2, 5, 10 and 15 mg/ml) were made to generate a calibration curve. Of each diluted protein sample (of the BSA and the cocktail), 200 μl of the supernatant was transferred into a 1.5 ml reaction tube. 800 μl BioQuant Biuret reagent was added and mixed thoroughly. From the same diluted protein sample, 500 μl was added to reaction tube containing a 10 KD filter. 200 μl of the effluent was transferred into a 1.5 ml reaction tube, 800 μl BioQuant Biuret reagent was added and mixed thoroughly. Next, all the mixtures (diluted protein sample before and after 10 KD filtration mixed with BioQuant) were incubated at room temperature for at least 30 minutes. The absorption of the mixtures was measured at 546 nm with a water sample used as a blank measurement. Dilutions of the cocktail that gave an absorption value at 546 nm within the range of the calibration line were used to calculate the total protein concentration of the cellulase cocktail samples via the BSA calibration line.

Enzymatic beta-glucosidase activity (WBDG) was determined at 37° C. and pH 4.4 using para-nitrophenyl-β-D-glucopyranoside as substrate. Enzymatic hydrolysis of pNP-beta-D-glucopyranoside resulted in release of para-nitrophenol (pNP) and D-glucose. Quantitatively released para-nitrophenol, determined under alkaline conditions, was a measure for enzymatic activity. After 10 minutes of incubation, the reaction was stopped by adding 1 M sodium carbonate and the absorbance was determined at a wavelength of 405 nm. Beta-glucosidase activity was calculated making use of the molar extinction coefficient of para-nitrophenol. A para-nitro-phenol calibration line was prepared by diluting a 10 mM pNP stock solution in acetate buffer 100 mM pH 4.40 0.1% BSA to pNP concentrations 0.25, 0.40, 0.67 and 1.25 mM. The substrate was a solution of 5.0 mM pNP-BDG in an acetate buffer (100 mM, pH 4.4). To 3 ml substrate, 200 μl of calibration solution and 3 ml 1M sodium carbonate was added. The absorption of the mixture was measured at 405 nm with an acetate buffer (100 mM) used as a blank measurement. The pNP content was calculated using standard calculation protocols known in the art, by plotting the OD₄₀₅ versus the concentration of samples with known concentration, followed by the calculation of the concentration of the unknown samples using the equation generated from the calibration line. Samples were diluted in weight corresponding to an activity between 1.7 and 3.3 units. To 3 ml substrate, preheated to 37° C., 200 μl of diluted sample solution was added. This was recorded as t=0. After 10.0 minutes, the reaction was stopped by adding 3 ml 1M sodium carbonate. The beta-glucosidase activity is expressed in WBDG units per gram enzyme broth. One WBDG unit is defined as the amount of enzyme that liberates one nanomol para-nitrophenol per second from para-nitrophenyl-beta-D-glucopyranoside under the defined assay conditions (4.7 mM pNPBDG, pH=4.4 and T=37° C.).

Concentrated pretreated carbohydrate material was made by incubating corn stover for 6.7 minutes at 186° C. Prior to the heat treatment, the corn stover was impregnated with H₂SO₄ for 10 minutes to set the pH at 2.3 during the pretreatment.

Enzymatic hydrolysis reactions were done in stirred, pH-controlled and temperature-controlled closed reactors with a working volume of 1 l. Each hydrolysis was done at pH 4.5 and at 62° C. The concentrated pretreated carbohydrate material was diluted with water to obtain a pretreated carbohydrate material with a final concentration of 17% (w/w) dry matter. Subsequently, the pH was adjusted to pH 4.5 with:

Experiment 1: 10% (w/w) NH₃ (aq) solution;

Experiment 2: 4 M potassium hydroxide solution;

Experiment 3: 4 M sodium hydroxide solution;

Experiment 4: 5 M calcium hydroxide solution.

For each experiment the same solutions were used to maintain the pH at 4.5 during the enzymatic hydrolysis.

The reactors used for enzymatic hydrolysis were stirred at 150 rpm for 18 hours, while the headspace was continuously refreshed by a flow of nitrogen (100 ml/min) at 62° C. Subsequently, the hydrolysis reactions were started by the addition of 2.5 mg Rasamsonia emersonii cellulase cocktail+300 WBDG/g dry matter. After 24 hours of hydrolysis, the nitrogen flow (100 ml/min) was exchanged by an air flow (100 ml/min) and the stirring speed was increased to 250 rpm, resulting in a dissolved oxygen (DO) level of 70% (i.e. 0.111 mol/m³) in the reaction mixture as measured by a DO-electrode. The total enzymatic hydrolysis time was 120 hours.

At the end of the hydrolysis, samples were taken for analysis which were immediately centrifuged for 8 minutes at 4000×g. The supernatant was filtered over 0.2 μm nylon filters (Whatman) and stored at 4° C. until analysis for sugar content as described below.

The sugar concentrations of the diluted samples were measured using an HPLC equipped with an Aminex HPX-87H column according to the NREL technical report NREL/TP-510-42623, January 2008. The results are presented in Table 1.

The results in Table 1 clearly show a higher glucose and xylose concentration when the pH of the pretreated carbohydrate material is controlled before and/or during enzymatic hydrolysis by adding a hydroxide of an alkali metal and/or a hydroxide of an alkaline earth metal (e.g. potassium hydroxide, sodium hydroxide or calcium hydroxide) to the pretreated carbohydrate material.

### Example 2

### pH Control in Enzymatic Hydrolysis of Wood

The effect of using different titrants to adjust and maintain the pH at a constant value during an enzymatic hydrolysis process for making hydrolysates useful for fermentative enzyme production process is shown in this example.

Rasamsonia emersonii cellulase cocktail (i.e. a whole fermentation broth) was produced according to the methods as described in WO 2011/000949. Moreover, Rasamsonia emersonii beta-glucosidase as described in WO2012/000890 was used in the experiments. The protein concentration of the cellulase cocktail and the enzymatic beta-glucosidase activity (WBDG) were determined as described in Example 1.

Pretreated carbohydrate material was made from poplar wood by bench scale steam explosion equipment with H₂SO₄ for 10 minutes at 180° C. (pH 1.75).

Prior to enzymatic hydrolysis, the pH of the pretreated carbohydrate material (containing 30% (w/w) dry matter) was adjusted to pH 4.5 using two different titrants (Experiment 1 and 2) as described below. The pretreated carbohydrate material was dilted with water to obtain a pretreated carbohydrate material with a final concentration of 10% (w/w) dry matter. Enzymatic hydrolysis reactions were done in stirred, pH-controlled and temperature-controlled closed reactors with a working volume of 1 l. Each hydrolysis was done at pH 4.5 and at 62° C.

Experiment 1: 10% (w/w) NH₃ (aq) solution;

Experiment 2: 4 M sodium hydroxide solution.

The hydrolysis reactions were started by the addition of 10 mg/g dry matter Rasamsonia emersonii cellulase cocktail+1200 WBDG/g dry matter. The total enzymatic hydrolysis time was 72 hours.

At the end of the hydrolysis, the obtained hydrolysates were filtered using a plate HS2000 depth filter and concentrated using a rotatory evaporator at 50° C. until a concentration of about 500 g total sugars per kg of concentrated hydrolysate as such. Thereafter, the concentrated hydrolysates were sterilised to make them ready for use in a fermentation process to produce a fungal cellulolytic enzyme cocktail. Sterilisation was done by autoclaving the hydrolysates for 10 minutes at 110° C. The results are presented in Table 2.

The results in Table 2 clearly show that the hydrolysate made by controlling the pH of the pretreated carbohydrate material before and/or during enzymatic hydrolysis by adding ammonia cannot be used in a fermentation process to make a fungal cellulolytic enzyme cocktail due to precipitation issues, while the hydrolysate made by controlling the pH of the pretreated carbohydrate material before and/or during enzymatic hydrolysis by adding a hydroxide of an alkali metal and/or a hydroxide of an alkaline earth metal (e.g. sodium hydroxide) is suitable for making a fungal cellulolytic enzyme cocktail in a fermentation process.

Similar results were also found when sterilisation was done by sterilizing the hydrolysates for 15 minutes at 120° C.

Similar results were also found when waste wood was used as concentrated pretreated carbohydrate material.

### Example 3

### pH Control in Enzymatic Hydrolysis of Wood

The experiment was done as described in Example 2 with the proviso that the pretreatment was done at pilot-scale at similar severity conditions, the enzymatic hydrolysis reactions were done in stirred, pH-controlled and temperature-controlled closed reactors with a working volume of 4 m³. The results are shown in Table 3.

Table 3 shows that the results of large-scale experiments are similar to the results of Example 2 that no precipitation issues were encountered when the pH of the pretreated carbohydrate material is controlled before and/or during hydrolysis by adding a hydroxide of an alkali metal and/or a hydroxide of an alkaline earth metal to the pretreated carbohydrate material. The hydrolysates obtained were when the pH of the pretreated carbohydrate material is controlled before and/or during hydrolysis by adding a hydroxide of an alkali metal and/or a hydroxide of an alkaline earth metal to the pretreated carbohydrate material were used in a fermentation process to make a fungal cellulolytic enzyme cocktail and were found to be readily fermentable.

TABLE 1
Glucose and xylose concentrations after 120 hours of
hydrolysis using different solutions for pH control.

| Experiment # | Glucose (g/L) | Xylose (g/L) | Glucose + Xylose (g/L) |
|---|---|---|---|
| 1 | 42.4 | 30.7 | 73.1 |
| 2 | 43.6 | 32.7 | 76.3 |
| 3 | 44.3 | 32.6 | 76.9 |
| 4 | 43.3 | 33.2 | 76.5 |

TABLE 2
Production of a fungal cellulolytic
enzyme cocktail with hydrolysates.

| | Precipitation of the | |
|---|---|---|
| | hydrolysate after | Hydrolysate suitable for |
| Experiment # | sterilisation | enzyme cocktail production |
| 1 | + | − |
| 2 | − | + |

TABLE 3
Production of a fungal cellulolytic enzyme
cocktail with hydrolysates on large-scale.

| | Precipitation of the | |
|---|---|---|
| | hydrolysate after | Hydrolysate suitable for |
| Experiment # | sterilisation | enzyme cocktail production |
| 1 | + | − |
| 2 | − | +* |

*Hydrolysate was successfully used in enzyme cocktail production

1. A process for preparation of an enzyme composition, comprising:
a) pretreating cellulosic material,
b) enzymatically hydrolysing the pretreated cellulosic material to obtain a hydrolysate,
c) fermenting the hydrolysate to produce the enzyme composition, and
a) optionally, recovering the enzyme composition,
wherein the pH of the pretreated cellulosic material is controlled before and/or during (b) by adding a hydroxide of an alkali metal and/or a hydroxide of an alkaline earth metal to the pretreated cellulosic material.

2. The process according to claim 1, wherein the obtained hydrolysate is concentrated before fermentation.

3. The process according to claim 2, wherein the concentrated hydrolysate is sterilized before fermentation.

4. The process according to claim 1, wherein the hydroxide of an alkali metal and the hydroxide of an alkaline earth metal are selected from the group consisting of aluminium hydroxide, barium hydroxide, calcium hydroxide, caesium hydroxide, potassium hydroxide, lithium hydroxide, magnesium hydroxide, sodium hydroxide, rubidium hydroxide, strontium hydroxide and any combination thereof.

5. The process according to claim 1, wherein the pH of the pretreated cellulosic material is controlled before and/or during (b) such that is from 3.0 to 6.5.

6. The process according to claim 1, wherein oxygen is added during (b).

7. The process according to claim 1, wherein the enzyme composition is produced by a fungus.

8. The process according to claim 1, wherein the enzyme composition comprises a whole fermentation broth of a fungus.

9. The process according to claim 1, wherein the enzyme composition comprises a cellobiohydrolase, an endoglucanase, a beta-glucosidase, an endoxylanase, a beta-xylosidase and a lytic polysaccharide monooxygenase.

10. The process according to claim 1, comprising a dry matter content in the hydrolysis from 10% to 40% (w/w).

11. The process according to claim 1, wherein the pH is measured before and/or during step (b).

12. A concentrated hydrolysate comprising 500-900 g sugars/kg dry matter hydrolysate and 0.5-3.5% (w/w) of a hydroxide of an alkali metal and/or a hydroxide of an alkaline earth metal.

13. A fermentation mixture comprising a hydrolysate, a fungus and 0.02-20 g of a hydroxide of an alkali metal and/or a hydroxide of an alkaline earth metal per kg fermentation mixture.
